(19) United States
(12) Patent Application Publication
(10) Pub. No.: US 2024/0059719 A1
(43) Pub. Date: Feb. 22, 2024

(54) GOLD COMPLEXES AS ANTICANCER AGENT

(71) Applicant: King Saud University, Riyadh (SA)

(72) Inventors: Haseeb Ahmad Khan, Riyadh (SA); Abdullah S. Alhomida, Riyadh (SA); Anvarhusein A. Isab, Dhahran (SA); Mansour K. Gatasheh, Riyadh (SA); Ali Al-Hoshani, Riyadh (SA); N Rajendra Prasad, Annamalai Nagar (IN)

(21) Appl. No.: 18/383,517

(22) Filed: Oct. 25, 2023

(51) Int. Cl. C07F 9/535; A61P 35/00

(52) U.S. Cl. CPC C07F 9/535; A61P 35/00

(57) ABSTRACT

The present invention relates to a gold complex having a compound of formula (I) as anticancer or chemotherapeutic agent. The present invention also relates to method of preparation of such complexes, pharmaceutical compositions containing such complexes and further extends to a method of treating or diagnosing a subject/patient suffering from cancer using such complexes.

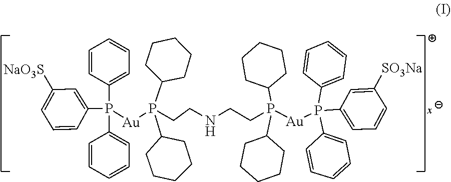

### FIELD OF THE INVENTION

The present disclosure generally relates to inorganic complexes. Specifically, the present disclosure relates to a gold complex having a compound of formula (I) as anticancer or chemotherapeutic agent. The present invention also relates to the preparation of the gold complex having a compound of formula (I) and composition comprising the gold complex of formula (I) for cancer treatment.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Cancer is one of the deadliest and costliest diseases and is the second leading cause of death worldwide. Among all cancer types, breast cancer is one of the most frequently diagnosed cancers and is the second leading cause of cancer-related mortality worldwide (Ferlay et al International Agency or Research on Cancer 2021; (available from: http://ci5.iarc.fr)). Statistics from the International Agency for Research on Cancer (IARC) indicate that in 2020 alone, approximately 2.3 million women were diagnosed with breast cancer and around 685,000 deaths were reported (Sung et al., CA Cancer J Clin. 2021; 71: 209-249). The pathogenesis of breast cancer is multifactorial and various factors such as age, genetics, exposure to estrogen, and lifestyle choices including diet, exercise, and alcohol consumption contribute to the development of this disease (Key et al., Lancet Oncol. 2001; 2 (3): 133-140). Recent advances in screening methods, early detection, and improved treatment modalities have resulted in better survival rates for individuals affected with breast cancer (Siegel et al., CA Cancer J Clin, 2020; 70: 7-30).

Chemotherapy drugs are powerful medications used to treat cancers by killing or impairing the growth of cancer cells. There are various types of chemotherapy drugs depending on their mode of action. For instance: doxorubicin, which is commonly used to treat breast, lung, ovarian, and thyroid cancers, works by intercalating DNA and inhibiting topoisomerase II enzyme, leading to cell death (Tacar et al., J Pharm Pharmacol. 2013; 65: 157-170). The alkylating agent, cyclophosphamide, which is used to treat lymphomas, leukemias, and solid tumors like breast and ovarian cancers, functions by cross-linking DNA strands to prevent cell proliferation (Emadi and Jones, Nat Rev Clin Oncol. 2009; 6: 638-647). Paclitaxel (Taxol) stabilizes microtubules and prevents their breakdown during cell division, leading to cell death in various malignancies such as breast, ovarian, and lung cancers (Rowinsky et al., J Natl Cancer Inst. 1990; 82: 1247-1259). Fluorouracil (5-FU), which is widely used to treat colorectal, breast, head and neck cancers, works by inhibiting thymidylate synthase, a key enzyme involved in DNA synthesis (Longley et al., Nat Rev Cancer. 2003; 3: 330-338),

Platinum-based drugs, such as cisplatin, carboplatin, and oxaliplatin, are widely used chemotherapeutic agents in the treatment of various types of cancers Dasari and Tchounwou, Eur J Pharmacol. 2014; 740: 364-378). The potential anticancer mechanisms of these drugs are based on typical interactions between drugs and cellular components, in particular, DNA and various enzymes involved in the synthesis and repair of DNA. Regarding the mechanism related to DNA binding and intra-strand crosslinking, the platinum drugs preferentially bind to purines (adenine and guanine) in DNA molecule, producing intra-strand crosslinks. Because of these crosslinks, the structure of DNA is distorted resulting in obstruction of DNA replication and transcription process.

Inhibition of DNA repair enzymes including nucleotide excision repair (NER) and mismatch repair (MMR) enzymes is another important mechanism behind anticancer effects of platinum-based drugs. By inhibiting these enzymes, platinum drugs potentially increase the cytotoxicity linked with the damage of DNA (Kelland L., Nature Rev Cancer. 2007; 7: 573-584). Another important mechanism is related to the induction of apoptosis due to the accumulation of damaged (unrepaired) DNA from the processes mentioned above, leading to cell cycle arrest and programmed cell death (apoptosis). This process involves the activation of pro-apoptotic proteins like BAX, p53, and caspases. Notwithstanding the clinical effectiveness of these mechanisms, platinum-based drugs are associated with numerous adverse effects due to their non-specific cytotoxicity such as nephrotoxicity of cisplatin or ototoxicity which may lead to irreversible hearing loss through damage to inner ear sensory hair cells or peripheral neuropathy resulting in motor and sensory deficit (Cavaletti and Marmiroli, Nature Rev Neural. 2010; 6: 657-666).

Historically, the medical application of gold is tracked back to 2500 BC as Chinese people relied on gold as an important therapeutic agent (Huaizhi and Yuantao, Gold Bull. 2001; 34: 24-29). Later on, antibacterial and anti-tubercular properties of gold compounds were discovered. Gold therapy was also found to alleviate joints pain, which led to the development of auranofin for treating the patients of rheumatoid arthritis (Sutton et al., J Med Chem., 1972; 15: 1095-1098). In recent years, gold complexes have gained attention due to their potential application in cancer treatment. Initially, the anticancer activity of the gold (I) complex [Au(dppe)2]Cl was evaluated in preclinical trials however due to certain toxicological concerns the study was halted.

Various studies have been conducted to understand the mechanisms and therapeutic potential of gold complexes in cancer treatment. For instance, Marzano et al (J Med Chem. 2007; 50: 4315-4321) demonstrated that gold (I) phosphine complexes can cause apoptosis in cancer cells. Similarly, Ott et at (Eur J Inorg Chem. 2010; 2010: 5076-5080) reported that a gold (I) N-heterocyclic carbene complex exhibited cytotoxic activity against human ovarian cancer cells. Given the significant impact of this disease, there is a critical need to develop innovative anticancer drugs that offer enhanced efficacy with minimal adverse effects. Continuous effort in researching novel therapeutic approaches is paramount in addressing this ongoing medical challenge.

There is therefore a need in the art to develop novel anticancer agents that is highly effective for the treatment or diagnosis of the cancer diseases with better safety profile.

### OBJECTS OF THE INVENTION

Primary objective of the present disclosure is to provide novel gold (I) complexes for effective treatment or diagnosis of cancer diseases.

Another objective of the present disclosure is to provide the gold (I) complexes for effective treatment of breast cancer.

Another objective of the present disclosure is to provide a method of preparation of the gold (I) complexes.

Another objective of the present disclosure is to provide a method of treating cancer using the gold (I) complexes.

### SUMMARY OF THE INVENTION

The present disclosure relates to a gold complex having a compound of formula (I) as anticancer or chemotherapeutic agent. The present invention also relates to method of preparation of such complexes, pharmaceutical compositions containing such complexes and further extends to a method of treating or diagnosing a subject/patient suffering from cancer using such complexes.

In an aspect, the present invention relates to gold complex having a compound of formula (I),

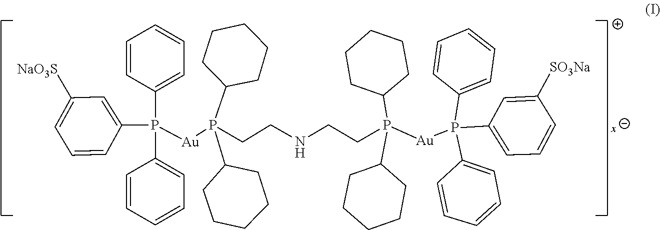

wherein:
X^{Θ} is PF₆ or BF₄.

In another aspect of the present invention, the gold complex is

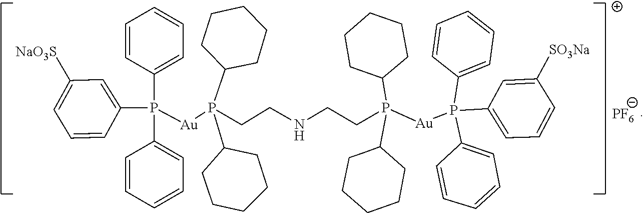

In another aspect, the present invention relates to method of preparing a gold complex having a compound of formula (I) comprising the steps of:
(a) reacting silver hexafluorophosphate (AgPF₆) with chloro[diphenyl(3-sulfonatophenyl)phosphine]gold(I), sodium salt hydrate to obtain gold hexafluorophosphate complex; and
(b) reacting the gold hexafluorophosphate complex with the ligand, bis(2-dicyclohexylphosphino)ethylamine to obtain the gold complex having a compound of formula (I).

In another aspect, the present invention relates to a pharmaceutical composition comprising a compound of formula (I) along with one or more pharmaceutically acceptable carrier.

In yet another aspect, the present invention relates to a method for treating cancer, comprising administering to a patient suffering from cancer an effective amount of a compound represented by formula (I), after suitable pre-clinical and clinical trials.

In another aspect of the present invention, the cancer is breast cancer, esophageal cancer, lung cancer, colon cancer, ovarian cancer, leukemia, renal cancer, melanoma cancer, prostate cancer, CNS cancer, carcinoma, lymphoma, blastoma, sarcoma, leukemia, gastric or stomach cancer, gastrointestinal cancer, pancreatic cancer, glioblastoma, cervical cancer, ovarian cancer, liver cancer, bladder cancer, cancer of the urinary tract, hepatoma, rectal cancer, colorectal cancer, endometrial or uterine carcinoma, salivary gland carcinoma, vulval cancer, thyroid cancer, hepatic carcinoma, anal carcinoma, and penile carcinoma.

The following extensive discussion of preferred embodiments will reveal several objects, features, characteristics, and advantages of the inventive subject matter.

### BRIEF DESCRIPTION OF THE FIGURES

FIG. 1 shows comparative cytotoxicity effect of the gold complex of the present invention, PBTDG and sorafenib at different concentrations in MCF-7 breast cancer cells.

FIG. 2 shows the comparative effects of PBTDG and erlotinib on cellular ATP levels in MCF-7 breast cancer cells at different concentrations.

FIG. 3 depicts the comparative effects of PBTDG and sorafenib on mitochondrial membrane potential depolarization in cancer cells; *P<0.01, **P<0.001 and ***P<0.001 versus control group.

FIG. 4 depicts the comparative effects of different concentrations of PBTDG and sorafenib on cellular apoptosis in cancer cells, measured by flow cytometric analysis.

FIG. 5 represents percent apoptosis (combined early and late apoptosis) induced by different concentrations of PBTDG and sorafenib in cancer cells. *P<0.05, **P<0.01 and ***P<0.001 versus control group (0 μM concentration).

FIG. 6 represents comparative effects of different concentrations of PBTDG and sorafenib on the generation of reactive oxygen species (ROS) in cancer cells, measured by flow cytometric analysis. *P<0.01 and **P<0.001 versus control group.

### DETAILED DESCRIPTION OF THE INVENTION

The embodiments herein and the various features and advantageous details thereof are explained more comprehensively with reference to the non-limiting embodiments that are detailed in the following description. Descriptions of well-known components and processing techniques are omitted so as to not unnecessarily obscure the embodiments herein. The examples used herein are intended merely to facilitate an understanding of the ways in which the embodiments herein may be practiced and to further enable those of skill in the art to practice the embodiments herein. Accordingly, the examples should not be construed as limiting the scope of the embodiments herein.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

As used in the description herein, the meaning of “a,” “an,” and “the” includes plural reference unless the context clearly dictates otherwise. Also, as used in the description herein, the meaning of “in” includes “in” and “on” unless the context clearly dictates otherwise.

As used herein, the terms “comprise”, “comprises”, “comprising”, “include”, “includes”, and “including” are meant to be non-limiting, i.e., other steps and other ingredients which do not affect the end of result can be added. The above terms encompass the terms “consisting of” and “consisting essentially of”.

The term “PBTDG” as used herein refers to the gold complex of the present invention represented by chemical structure below.

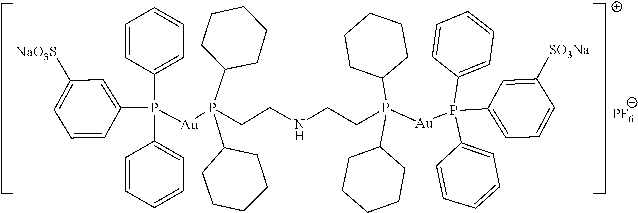

The present disclosure relates to a gold complex having a compound of formula (I) as anticancer or chemotherapeutic agent. The present invention also relates to method of preparation of such complexes, pharmaceutical compositions containing such complexes and further extends to a method of treating or diagnosing a subject/patient suffering from cancer using such complexes.

In an embodiment, the present invention relates to a gold complex having a compound of formula (I),

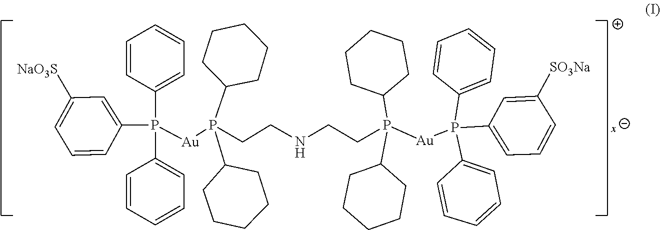

wherein:
X^{Θ} is PF₆ or BF₄.

In another embodiment of the present invention, the X^{Θ} is PF₆.

In another embodiment of the present invention, the X^{Θ} is BF₄.

In yet another embodiment, the present invention relates to a method of preparing a gold complex having a compound of formula (I) comprising the steps of:
(a) reacting silver hexafluorophosphate (AgPF₆) with chloro[diphenyl(3-sulfonatophenyl)phosphi tie] gold(I), sodium salt hydrate to obtain gold hexafluorophosphate complex; and
(b) reacting the gold hexafluorophosphate complex with the ligand, bis(2-dicyclohexylphosphino)ethyl amine to obtain the gold complex having a compound of formula (I).

In another embodiment of the present invention, the silver hexafluorophosphate (AgPF₆) can be dissolved in solvents such as ethanol, methanol and isoproponal,

In another embodiment of the present invention, the chloro[diphenyl(3-sulfonatophenyl)phosphine] gold(I), sodium salt hydrate can be dissolved in solvents such as ethanol, methanol and isoproponal.

In another embodiment of the present invention, the process of preparing the gold complex is carried out at a room temperature.

In yet another embodiment, the present invention relates to pharmaceutical composition comprising the gold complex have a compound of formula (I) in combination with one or more pharmaceutically acceptable excipients, additives or carriers. The excipients are preferably inert and in any event non-toxic to the subject/patient for which the pharmaceutical composition is intended.

In another embodiment of the present invention, the pharmaceutical excipients, additives and carriers may include, but are not limited to including, proteins, peptides, amino acids, lipids, and carbohydrates (e.g. sugars, including monosaccharides, di-, tri-, tetra-, and oligosaccharides; derivatized sugars such as alditols, aldonic acids, esterified sugars and the like; and polysaccharides or sugar polymers), which can be present singly or in combination.

According to another aspect of the present invention, there is provided the use of a gold (I) complex according to formula (I), as herein before described, in the manufacture of a medicament for the treatment, diagnosis and/or prevention of cancer.

The invention further provides for the use of gold (I) complex according to formula (I), as herein before described, for selectively inhibiting the activity of cancer cells.

In terms of the present invention, the cancer may be selected from the group consisting of breast cancer, oesophageal cancer, lung cancer, colon cancer, ovarian cancer, leukaemia, renal cancer, melanoma cancer, prostate cancer, CNS cancer, carcinoma, lymphoma, blastoma, sarcoma, leukemia, gastric or stomach cancer, gastrointestinal cancer, pancreatic cancer, glioblastoma, cervical cancer, ovarian cancer, liver cancer, bladder cancer, cancer of the urinary tract, hepatoma, rectal cancer, colorectal cancer, endometrial or uterine carcinoma, salivary gland carcinoma, vulval cancer, thyroid cancer, hepatic carcinoma, anal carcinoma and penile carcinoma.

It will be appreciated that the scope of the present invention is not limited to the cancer types identified herein.

In another aspect of the invention, there is provided a method of treating or diagnosing a subject/patient suffering from cancer comprising administering to a subject patient in need thereof an effective amount of the gold (I) complex according to formula (I), as herein before described.

The present invention also provides a method of inhibiting metastasis of cancer comprising administering to a subject in need thereof an effective amount of gold (I) complex according to formula (I), as described herein.

The present invention further provides a method of reducing cell growth of cancer comprising administering to a subject in need thereof an effective amount of the gold (I) complex according to formula (I), as described herein.

In another embodiment, the pharmaceutical composition described herein may be delivered by a variety of suitable drug delivery systems known and described in the art. Non-limiting examples of drug delivery systems and technologies for administering the pharmaceutical composition of the present invention in order to achieve the desired therapeutic effect include, but are not limited to, oral, nasal, and topical administration, administration by way of inhalation, administration by way of injection and administration by way of nano-biotechnology pharmaceutical delivery systems and devices.

The present invention further provides for the pharmaceutical composition, as described herein, to optionally include a therapeutic agent.

The present invention yet further provides for the pharmaceutical composition, as described herein, to optionally be administered together with an additional pharmaceutical preparation or a therapeutic agent.

According to a further aspect of the present invention, there is provided a composition including the gold (I) complex according to formula (I), as identified herein, for use as a diagnostic agent.

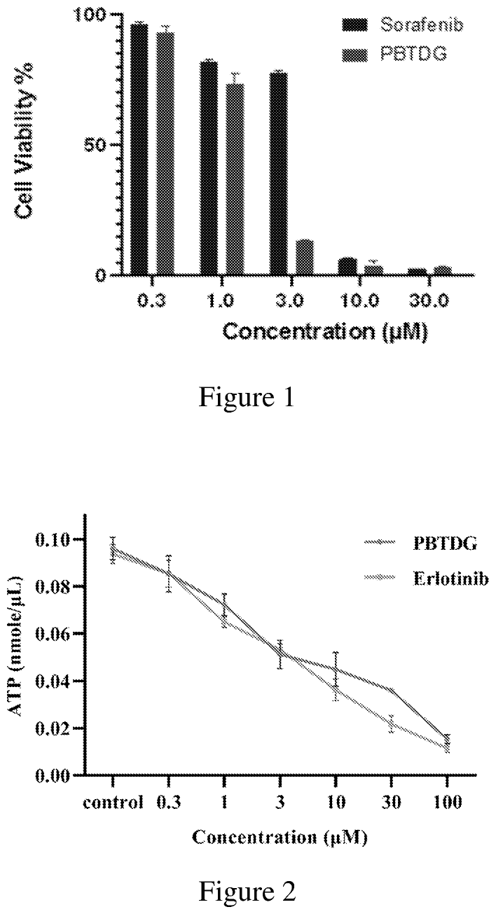

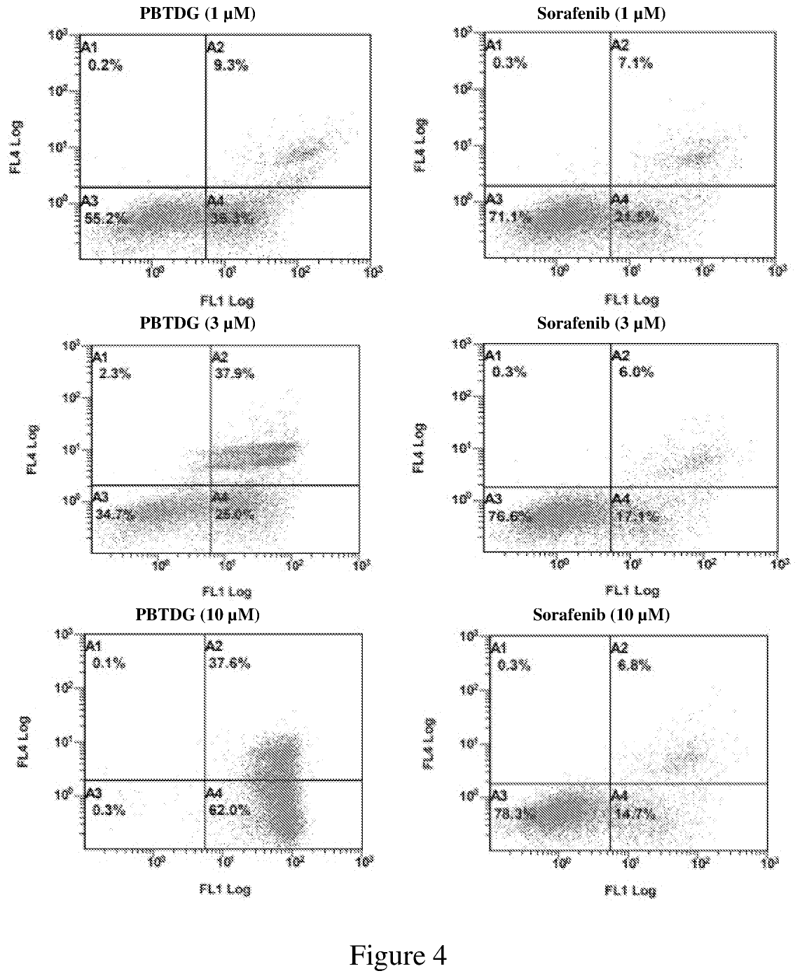

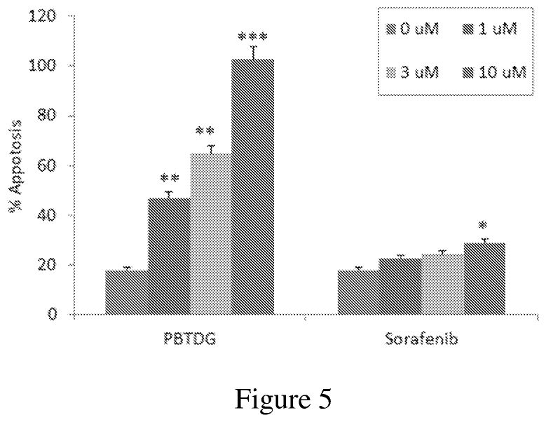

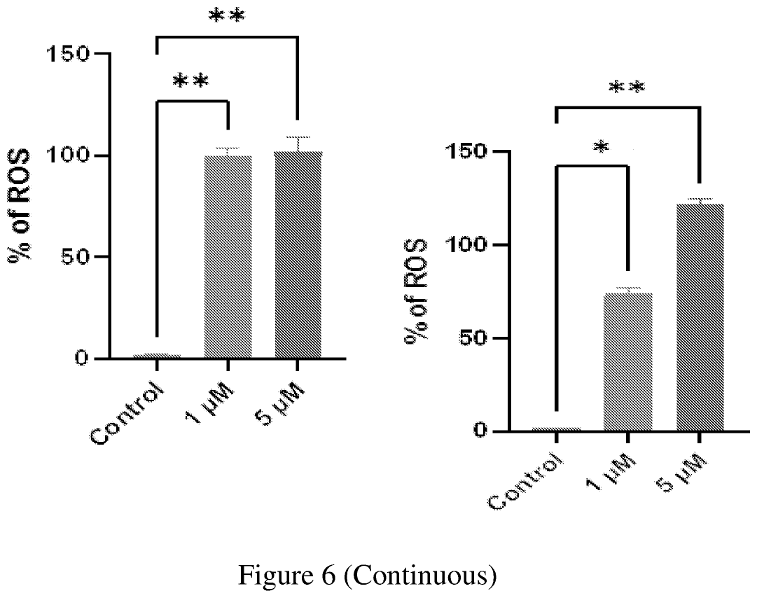

According to the present invention, the gold (I) complex, PBTDG, exhibited anticancer effects in breast cancer cells. The cytotoxic effects of PBTDG were significantly greater than sorafenib, which is a known anticancer drug for solid tumors (FIG. 1). The IC₅₀ value of the gold(I) complex, PBTDG (1.48 μM) in MCF-7 cells, was found to be much lower (more potent) than IC₅₀ values of two gold(I) complexes, 4a (7.62 μM) and 4b (4.70 μM), reported earlier by Hague et al (2016). The gold (I) complex PBTDG dose-dependently reduced cellular ATP levels (FIG. 1) and disrupted the mitochondrial membrane potential (FIG. 3), PBTDG exerted anti-proliferative effect by inducing apoptosis in cancer cells (FIG. 4). Also observed significant increase in the production of ROS in cancer cells treated with PBTDG (FIG. 6).

In another embodiment of the present invention, the anticancer activity of PBTDG at lower concentrations indicates its great potential for cost-effective cancer chemotherapy. The anti-proliferative capability of PBTDG is associated to the disruption of mitochondrial energy metabolism, generation of ROS and induction of apoptotic pathway. The selective toxicity of gold complexes against cancer cells, owing to their altered metabolism and higher reliance on mitochondrial energy production compared to healthy cells has opened promising avenues for novel therapies for cancer.

The present disclosure may be more fully understood by reference to the following examples:

### EXAMPLES

### Synthesis of Gold (I) Complex, PBTDG of the Present Invention

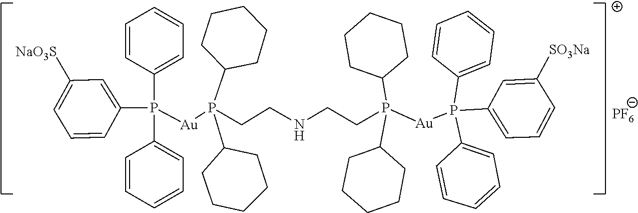

AgPF₆ (0.127 g, 0.5 mmol) dissolved in 5.0 mL of ethanol was added to chlorodiphenyl(3-sodiumsulfonatophenyl)phosphane)]gold(I), sodium salt hydrate (0.2984 g, 0.5 mmol) in 15.0 mL methanol. The mixture was stirred for 30 minutes at room temperature and then filtered to remove the white precipitate of AgCl. To the filtrate, bis(2-dicyclohexylphosphino)ethylamine (1164 g, 0.25 mmol) was added and the mixture were stirred for additional 1 hour and filtered. The clear colorless solution was kept in an undisturbed area for 3-5 days to obtain the white solid material. The solid material obtained was washed with dichloromethane and diethyl ether three times (5.0 mL). The complex was then recrystallized from acetonitrile solution.

¹NMR chemical shifts for gold complex, PBTDG in CDCl₃.

| | | | C4, 4′ | C4, 4′ | C5, 5′ | C5, 5′ | C6, 6′ | C6, 6′ | Aromatic | |
|---|---|---|---|---|---|---|---|---|---|---|
| C1 | C2 | C3 | eq | ex | eq | ex | eq | ex | Hs | NH |
| 3.40 | 2.51 | 1.38 | 2.29 | 1.22 | 1.91 | 1.91 | 1.74 | 1.26 | 7.56, 8.1, | 3.19 |
| | | | | | | | | | 8.53 | |

¹³C and ³¹P NMR chemical shifts for the gold complex, PBTDG, in CDCl₃.

| C1 | C2 | C3 | C4, | C5 | C6 | Aromatic Cs | ³¹P |
|---|---|---|---|---|---|---|---|
| 45 | 25.7 | 34.1 | 26.5 | 30.0 | 29.2 | 128.5, 129.8, 131.7, 134.1 | 39.9, 49.3 |

### Biological Studies

### Cell Viability Analysis

MTT (3-(4,5-dimethylthiazol-2-yl)-2,5-diphenyltetrazolium bromide) assay was used for cell viability analysis. MCF-7 breast cancer cells were seeded in a cell culture plate (96-well) at 10⁴ cells per well in 200 μl of Dulbecco's modified Eagle medium (DMEM). The cells were treated with Sorafenib and PBTDG at serial concentrations of 0.3, 1.0, 3.0, 10.0, and 30.0 μM for 24 hours. MTT solution (20 μl of 5 mg/mL) was added to each well of the micro plate, which were then incubated in a CO₂ incubator at 37° C. for 3 hours. After incubation, the culture medium was removed and 100 μL of isopropanol were added to each well of the micro plate and the absorbance was recorded at 570 nm against reagent blank. The cell viability was calculated using the formula:

Cell viability %=100*(Absorbance sample)/(Absorbance blank)

The results of cytotoxicity showed a direct relation between cellular death and the concentration of PBTDG and sorafenib, with the IC₅₀ values of 1.48 μM and 4.45 μM, respectively. At lower concentration, particularly at 3.0 μM, PBTDG was found to be more toxic than sorafenib for MCF-7 cells, whereas their toxicities were comparable at 30 μM (FIG. 1).

### Intracellular ATP Analysis

The intracellular ATP levels were determined after the exposure of PBTDG and erlotinib (0-100 μM) to MCF-7 cells for 12 h using a colorimetric ATP assay kit (Sigma-Aldrich, USA) according to the manufacturer instructions. The unknown concentrations of the test samples were calculated using the linear standard curve generated from the concentration of 0-12 nmole ATP. Equal amounts of Mn₃O₄ nanoparticles and NDG were milled using a Fritsch Pulverisette P7 planetary ball mill (Idar-Oberstein, Germany). The nanomaterials powder and stainless steel balls (5 mm diameter) with the ball to powder weight ratio of 1:1 were introduced into the stainless steel container. The milling of the powder was performed for 16 h, with intermittent pausing of milling process at regular intervals.

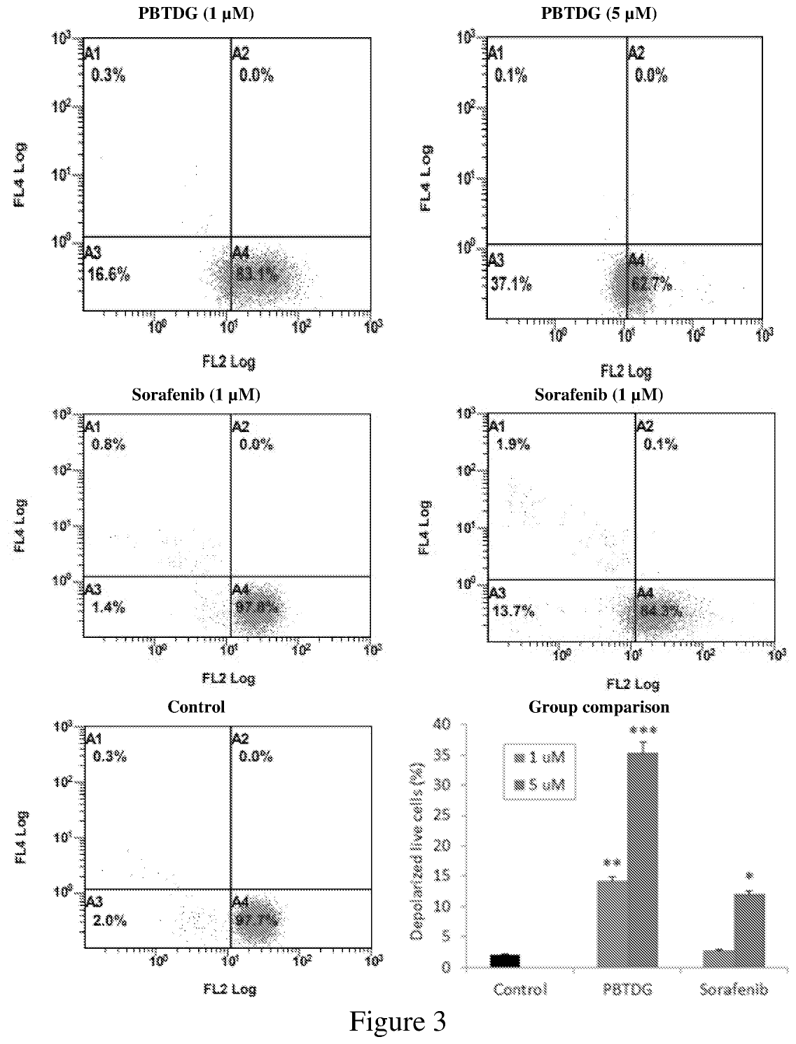

The intracellular ATP levels were determined in MCF-7 cells following the exposure of different concentrations of PBTDG and a positive control, erlotinib (0-100 μM). The control cells (without drug exposure) showed high levels of cellular ATP (FIG. 2). The concentrations of ATP depleted after the exposure of PBTDG or erlotinib. The drug-induced depletion of ATP was directly proportional to drug concentration (FIG. 2).

### Mitochondrial Membrane Potential Analysis

Muse MitoPotential Flow Cytometry Kit (Luminex, IL, USA) was used for measuring mitochondrial membrane potential (MMP). MCF-7 cells were seeded (15000 cells per well) in a 6-wells culture plate. The cells were allowed to culture for 24 hours at 37° C. and 5% CO₂ environment, and then exposed to Sorafenib or PBTDG at concentrations of 1 and 5 μM for another 24 hours. A negative control (DMSO) was run in parallel. Finally, the cells were stained with Mito Potential reagents provided in the kit, following the manufacturer's instructions, and then analyzed by flow cytometry.

The mitochondrial membrane was significantly depolarized by PBTDG as compared to positive control sorafenib (FIG. 3). The depolarization caused by higher concentration (5.0 μM) of PBTDG and sorafenib was found to be 35.4% and 12.0%, respectively. The low concentration (1.0 μM) of PBTDG and sorafenib depolarized the mitochondrial membrane by 14.2% and 2.7%, respectively (FIG. 3).

### Apoptosis Analysis

The apoptotic effects of Sorafenib and PBTDG were evaluated on MCF-7 breast cancer cells, using Muse® Annexin V Live & Dead Cell Kit (Luminex, IL, USA). The cells were seeded in 6-wells plates at 15000 cells per well and the plates were incubated for 24 hours at 37° C. and 5% CO₂ environment. Then the cells were exposed to sorafenib and PBTDG at the concentrations of 1.0, 3.0, and 10.0 μM for 24 hours. For negative control, diluted solution of DMSO was used so as the final DMSO concentration in each well was <0.1%. The cells were then stained with Annexin V-FITC and Dead Cell reagents according to manufacturer's instructions. The percentage of apoptotic cells was determined by flow cytometry.

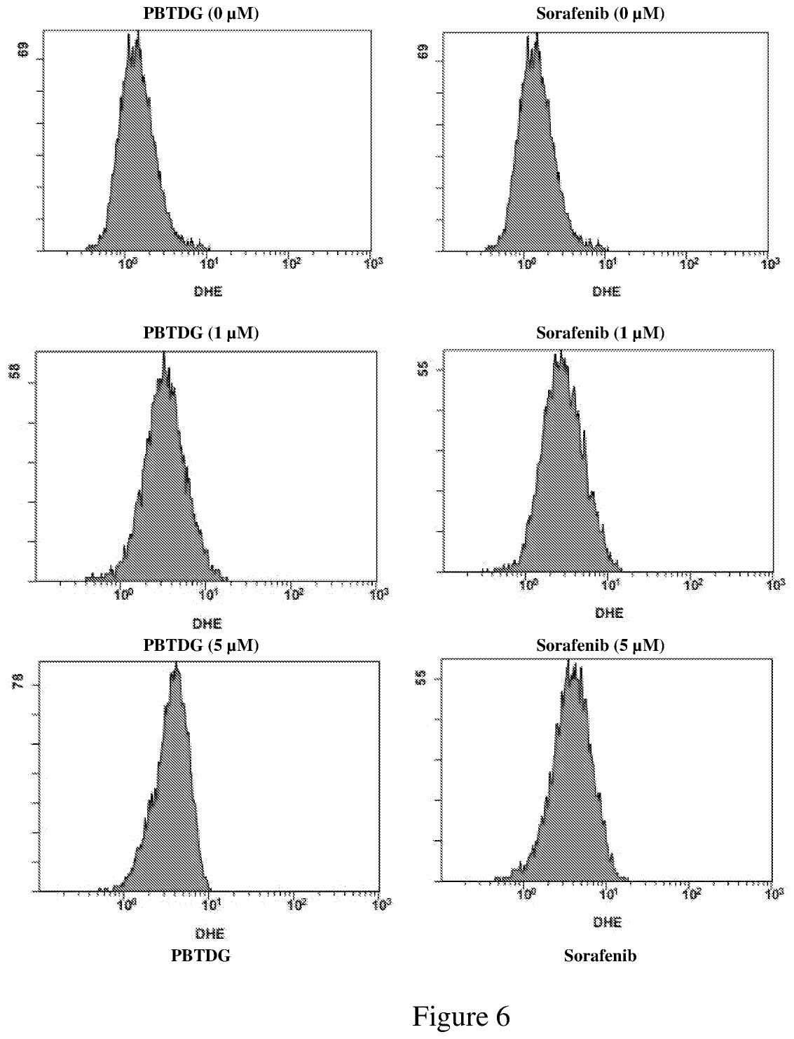

The results of apoptosis analysis showed that PBTDG induced 2.6 folds, 3.6 folds, 5.7 folds apoptosis for 1 μM, 3 μM., and 10 μM concentrations, respectively (FIG. 4). While the induction of apoptosis for sorafenib was found to be 1.2-folds (1 μM), 1.3-folds (3 μM) and 1.6-folds (10 μM). These findings clearly indicate that PBTDG induced significantly higher apoptotic effects as compared to anti-cancer drug, sorafenib (FIG. 5).

### Reactive Oxygen Species Analysis

For the analysis of reactive oxygen species (ROS), MCF-7 cells were seeded (1.5×1.04 cells/well) in a 6-well plate and allowed to grow for 24 h at 37° C. under the environment of 5% CO2 and 95% humidity. After incubation, MCF-7 cells were treated with the gold complex PBTDG and sorafenib at final concentrations of 1 and 5 μM for 24 h. DMS( )served as a negative control. The final DMSO concentration in each well was less than 0.1%. After harvesting the cells, they were stained with Muse® Oxidative Stress Kit (Luminex, IL, USA) following the manufacturer's instructions. The percentage of cells with oxidative stress was measured by flow cytometry analysis.

Effects of PBTDG and sorafenib were investigated on the generation of reactive oxygen species (ROS) in MCF-7 cancer cells. The low concentration of PBTDG (1 μM) induced the ROS generation by 99.83% which was significantly higher to the ROS generation caused by 1 μM of sorafenib (73.76%). The ROS induction caused by higher concentration (5 μM) of PBTDG and sorafenib were 104.95% and 122.11%, respectively (FIG. 6).

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

### Advantages of the Invention

The present invention provides a gold complex of formula (I) as effective for anticancer treatment, particularly, the complex for breast cancer treatments.

The present disclosure provides a gold complex of formula (I) that shows significantly greater cytotoxic &Teas.

1. A gold complex having a compound of formula (I): 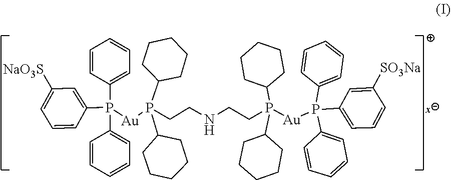
wherein:
X^{Θ} is PF₆ or BF₄.

2. The gold complex as claimed in claim 1, wherein X^{Θ} is PF₆.

3. The gold complex as claimed in claim 1, wherein the compound is:

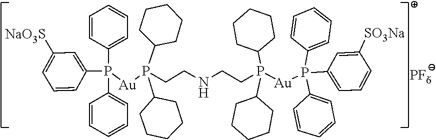

4. A process for the preparation of the complex as claimed in claim 1, wherein the process comprises the steps of:
(a) reacting silver hexafluorophosphate (AgPF₆) with chloro[diphenyl(3-sulfonatophenyl)phosphine] gold(I), sodium salt hydrate to obtain gold hexafluorophosphate complex; and
(b) reacting the gold hexafluorophosphate complex with the ligand, bis(2-dicyclohexylphosphino)ethyl amine to obtain the gold complex having a compound of formula (I).

5. A pharmaceutical composition comprising a therapeutically effective amount of the gold complex as claimed in claim 1 along with one or more pharmaceutically acceptable carriers.

6. A method for treating cancer, comprising administering to a patient suffering from cancer an effective amount of a compound of formula (I) as claimed in claim 1.

7. The method according to claim 6, wherein the treating comprises selectively inhibiting the activity of cancer cells.

8. The method according to claim 6, wherein the cancer is selected from the group consisting of breast cancer, esophageal cancer, lung cancer, colon cancer, ovarian cancer, leukemia, renal cancer, melanoma cancer, prostate cancer, CNS cancer, carcinoma, lymphoma, blastoma, sarcoma, leukemia, gastric or stomach cancer, gastrointestinal cancer, pancreatic cancer, glioblastoma, cervical cancer, ovarian cancer, liver cancer, bladder cancer, cancer of the urinary tract, hepatoma, rectal cancer, colorectal cancer, endometrial or uterine carcinoma, salivary gland carcinoma, vulval cancer, thyroid cancer, hepatic carcinoma, anal carcinoma, and penile carcinoma.

9. The method according to claim 6, wherein the cancer is breast cancer.

10. The method according to claim 6, wherein the treating comprises inhibiting metastasis of cancer.

11. The method according to claim 6, wherein the treating comprises reducing cell growth of cancer.
